(19) United States
(12) Patent Application Publication
(10) Pub. No.: US 2021/0228772 A1
(43) Pub. Date: Jul. 29, 2021

(54) BIOSCAFFOLD COMPOSITIONS OF MATTER

(71) Applicant: Mentor Worldwide LLC, Irvine, CA (US)

(72) Inventors: Hector Javier Toro Estrella, Irvine, CA (US); Orquidea Helen Garcia, Irvine, CA (US)

(21) Appl. No.: 17/053,409

(22) PCT Filed: May 6, 2019
(86) PCT No.: PCT/US2019/030959

§ 371 Date: Nov. 6, 2020

Related U.S. Application Data
(60) Provisional application No. 62/668,197, filed on May 7, 2018.

(51) Int. Cl. A61L 27/36; B33Y 70/10; B33Y 80/00; A61L 27/26; A61L 27/24; A61L 27/56; A61L 27/18

(52) U.S. Cl. CPC A61L 27/3633; B33Y 70/10; B33Y 80/00; A61L 27/26; C08L 67/04; A61L 27/56; A61L 27/18; A61L 2300/426; A61L 27/24

(57) ABSTRACT

The embodiments disclosed herein relate to acellular matrix bioscaffold compositions comprising 1) a natural or synthetic polymer or blend of polymers; 2) collagen I (Col-1); and 3) collagen III (Col-3), wherein the ratio of Col-1 to Col-3 is between 0.5 and 3.5, wherein said bioscaffold is synthetic.

### CROSS-REFERENCE TO RELATED APPLICATION

This application is an international application claiming the benefit of priority from U.S. Provisional Application No. 62/668,197 filed on May 7, 2018, the entirety of which is incorporated herein by reference.

### FIELD

The embodiments disclosed herein are generally directed towards compositions and materials used to form porous extracellular structures and/or bioscaffold structures that can be implanted into a patient. More specifically, there is a need for bioscaffold compositions and materials that minimize immunogenic response in a host while providing a structural framework for tissue regrowth.

### BACKGROUND

Current surgical trends lean towards bundled, minimally invasive and ambulatory procedures, and thus patient and physician preference continues to drive the adoption of lumpectomies and direct to implant procedures for the treatment of breast and other cancers. Following resection of the mastectomy specimen, patients are often left with poorly vascularized, thin or compromised tissue and/or skin flaps, necessitating the need for tissue reinforcement in order to complete implant based reconstruction. Similarly, following resection of the lumpectomy specimen, patients are often left with what is commonly known as a “shark bite” deformity, in which the remaining skin sinks into the void left by the resected tissue, leaving the patient with a permanent deformity. There are a number of conventional material options that are available to provide tissue reinforcement or fill-in the resected tissue to reconstruct the native form and shape of the organ or body section that the resected tissue was taken from. Examples include, fat (or other) grafted tissue fillers, pure artificial polymer-based scaffolds or acellular dermal matrix (ADM) bioscaffolds obtained from decellularized human, bovine, or porcine dermis can all be used. However, these conventional materials have a number of shortcomings as they can cause undesirable immunogenic responses in the patient (as is the case with ADM), do not provide sufficient support, protection and reinforcement to the dermis layer and cannot provide for robust cellular infiltration/remodeling response post implantation.

Besides producing adverse biological responses, conventional bioscaffolds often fail to provide optimal compositions of extracellular matrix proteins (e.g., collagen, elastin, laminin, cytokines, polysaccharides, growth factors, etc.) that help promote cell ingrowth into the bioscaffolds and thus boosting overall tissue regeneration which can help in patient recovery and reducing overall scarring.

Thus, a need exists for an implantable acellular scaffold that contains native extracellular matrix (ECM) proteins that can elicit a robust regenerative response (while minimizing undesirable immunogenic responses and other concerns discussed herein) from the host. This scaffold will not only fill the void post-surgery, but will also encourage host cell ingrowth, regeneration, repair, and be capable of serving as a scaffold for potential future grafts (i.e., fat grafts) as the surgeon deems appropriate.

### SUMMARY

In one aspect, an acellular matrix bioscaffold composition is provided, the composition comprising 1) a natural or synthetic polymer; 2) collagen I (Col-1); and 3) collagen III (Col-3), wherein the ratio of Col-1 to Col-3 is between about 0.5 and about 3.5, wherein said bioscaffold is synthetic. The ratio of Col-1 to Col-3 may be between about 0.9 and about 2.3. The ratio of Col-1 to Col-3 may be about 1. The polymer may be poly(ethylene glycol) (PEG), poly(lactide-co-clycolide) (PLGA), polycaprolactone (PCL), poly(l-lactic acid) (PLLA), alginate, hyaluronic acid, gelatin, soy protein, fibrinogen, chitosan, dextran, or starch. The polymer may be polycaprolactone (PCL). The polymer can be a blend of polymers.

In another aspect, an acellular matrix bioscaffold composition is provided, the composition comprising 1) a natural or synthetic polymer; 2) collagen I (Col-1); 3) collagen III (Col-3), and 4) polycaprolactone (PCL), wherein said bioscaffold is synthetic and the ratio of Col-1 to Col-3 is between about 0.5 and about 3.5. The composition may further include an antimicrobial agent. The composition may further include a growth factor, cytokine or other bioactive molecule. The polymer can be a blend of polymers.

In another aspect, an acellular matrix bioscaffold composition is provided, the composition comprising 1) a polymer, 2) collagen I (Col-1), and 3) collagen III (Col-3), wherein the ratio of Col-1 to Col-3 is between about 0.9 and about 2.3, with the proviso that the bioscaffold does not comprise the bioactive molecules present in cadaveric acellular dermal matrix (ADM). The composition may further comprise an antimicrobial agent. The composition may further comprise a growth factor, cytokine or other bioactive molecule.

The compositions may further include extracellular matrix proteins selected from the group consisting of laminin, fibronectin, elastin, glycosaminoglycans, and combinations thereof. The bioscaffold may have pores of about 100 to 500 microns, or about 50 to about 1000 microns.

In another aspect, a method for producing a bioscaffold structure is provided, the method comprising providing a polymer; providing a collagen-containing composition; combining the polymer, and collagen containing composition to form a first composition; lyophilizing the first composition to form a second composition; and crosslinking the collagen-containing composition and the polymer in the second composition to form the bioscaffold structure. In various embodiments, the polymer is a blend of polymers.

Additional aspects will be evident from the detailed description that follows, as well as the claims appended hereto and the drawings.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is an overview of the bioscaffold technology. Representative potential micro and macro structures are illustrated to demonstrate putative construct configurations, in accordance with various embodiments.

FIG. 2 illustrates a fused deposition modeling manufacturing process for producing a bioscaffold structure, in accordance with various embodiments.

FIG. 3 is a flowchart illustrating a process for producing a bioscaffold structure, in accordance with various embodiments.

FIG. 4 illustrates a fused deposition modeling manufacturing process for producing a bioscaffold structure, in accordance with various embodiments.

FIG. 5 is a flowchart illustrating a process for producing a bioscaffold structure, in accordance with various embodiments.

FIG. 6 illustrates a melt electrowriting/electrospinning based manufacturing process for producing a bioscaffold structure, in accordance with various embodiments.

FIG. 7 is a flowchart illustrating a process for producing a bioscaffold structure, in accordance with various embodiments.

FIG. 8 is a flowchart illustrating a process for producing a bioscaffold structure, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

This specification describes exemplary embodiments and applications of the disclosure. The disclosure, however, is not limited to these exemplary embodiments and applications or to the manner in which the exemplary embodiments and applications operate or are described herein. Other embodiments, features, objects, and advantages of the present teachings will be apparent from the description and accompanying drawings, and from the claims. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, a tray, a baseplate, a separate metal structure, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

As used herein, “extracellular”, as used in reference to, for example, “extracellular material”, “extracellular structure”, “extracellular matrix”, “extracellular construct”, and “extracellular component”, denotes the characteristic of existing outside the cell and can refer to a synthetic or natural material. Examples of materials that are extracellular include synthetic and natural polymers; metabolites; ions; various proteins and non-protein substances (e.g. DNA, RNA, lipids, microbial products, etc.) such as collagens, proteoglycans, hormones, growth factors, cytokines, chemokines; various enzymes including, for example, digestive enzymes (e.g., Trypsin and Pepsin), extracellular proteinases (e.g., matrix metalloproteinases, a disintegrin and metalloproteinase with thrombospondin motifs (ADAMTSs), Cathepsins) and antioxidant enzymes (e.g., extracellular superoxide dismutase); proteolytic products; extracellular matrix proteins (such as elastin, glycosaminoglycans (GAGs), laminin, fibronectin, etc.), selected cell populations, small molecules and small molecule inhibitors, antibiotics, antimicrobials, nanoparticles, mesoporous silica, silk fibroin, enzymatic degradation sites; anti-fibrotic agents such as anti-transforming growth factor beta (anti-TGF-β) and anti-tumor necrosis factor alpha (anti-TNF-α); pro-angiogenic agents such as vascular endothelial growth factor (VEGF) and placental growth factor (PlGF); and factors affecting adipogenesis and proliferation such as insulin-like growth factor 1 (IGF-1) and Dexamethasone.

As used herein, “bioink” denotes any bioactive, bioprintable, naturally or artificially derived material that mimics an extracellular matrix environment to support the adhesion, proliferation, and differentiation of living cells, and can be deposited as filaments, fibers or fibrils or droplets during an additive manufacturing process.

As used herein, “bioscaffold” denotes a biocompatible and bioresorbable structure used in tissue engineering that is capable of being implanted in the body in order to promote cell adhesion and tissue regeneration, often for injury recovery. A bioscaffold can be used, for example, in the areas of bone, cartilage, skin, organ, tissue area/volume (e.g., breast tissue), and muscle regeneration.

As used herein, “angular range” denotes a range of angles by which two objects can be placed relative to each other.

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

Unless otherwise defined, scientific and technical terms used in connection with the present teachings described herein shall have the meanings that are commonly understood by those of ordinary skill in the art.

This disclosure relates to novel acellular matrix bioscaffold compositions that may be used in a number of anatomical reconstruction procedures (e.g., breast reconstruction, breast augmentation). These novel compositions are suitable for three-dimensional printing of flat sheets, molded sheets, and/or solid 3D constructs. The bioscaffold composition will provide cushioning and structural support for other tissues, supplemental support, protection, reinforcement and covering within the breast, other organs, or other anatomy and surrounding tissue, while stimulating host cell remodeling. The bioscaffold composition may be biodegradable or readsorbable. It will allow plastic and reconstructive surgeons to support, repair, elevate, and reinforce deficiencies where weakness or voids exist in the breast, other organs, or other anatomy and surrounding tissues that requires the addition of material to obtain the desired surgical outcome.

The bioscaffold composition will also allow for the repair of fascial defects within the breast, other organs, or other anatomy and surrounding tissues that require the addition of a reinforcing or bridging material to obtain a desired surgical result. The bioscaffold composition may be printed in configurations including, but not limited to, flat sheets, molded sheets/constructs that provide a hand in glove fit for breast implants or other medical devices, anatomy or physiology within the breast pocket, other organs, or other anatomy, or as a lumpectomy defect implant within the breast, other organs, or other anatomy. Surprisingly, the inventors have discovered that the bioscaffold composition described in this disclosure will minimize host immunogenic response. The immunogenic response of the host may be reduced or minimized, for example, because the bioscaffold contains only known and desired components. Unlike current tissue offerings, which contain molecular moieties that may prove deleterious to the healing process and trigger a “graft vs. host response,” the various bioscaffold compositions described herein allow for the control of components and allow for standardization of clinical outcomes within and among patients. The inventors have also discovered optimal base ratios of Collagen-1 (Col-1) to Collagen-3 (Col-3) that provide increased support, cushioning, protection, reinforcement, and covering when compared against conventional bioscaffold composition offerings. Thus, the various bioscaffold compositions disclosed herein provide controlled elasticity and tensility in relation to currently available adult and elderly derived acellular dermal matrices.

The present disclosure also relates to a new generation of printable acellular matrix bioscaffold compositions with unique printability into 2D and 3D shapes and ability to support tissue and organ growth. Besides the advantages described above, the bioscaffold compositions described herein are an improvement over the prior art because they also minimize concerns over donor availability and the use of conventional tissue fillers (e.g., fat, pure polymer-based extracellular matrix, ADM, etc.), alleviate increasing costs, eliminate concerns over tissue quality, variability and contamination potential, and provide a minimally immunogenic solution.

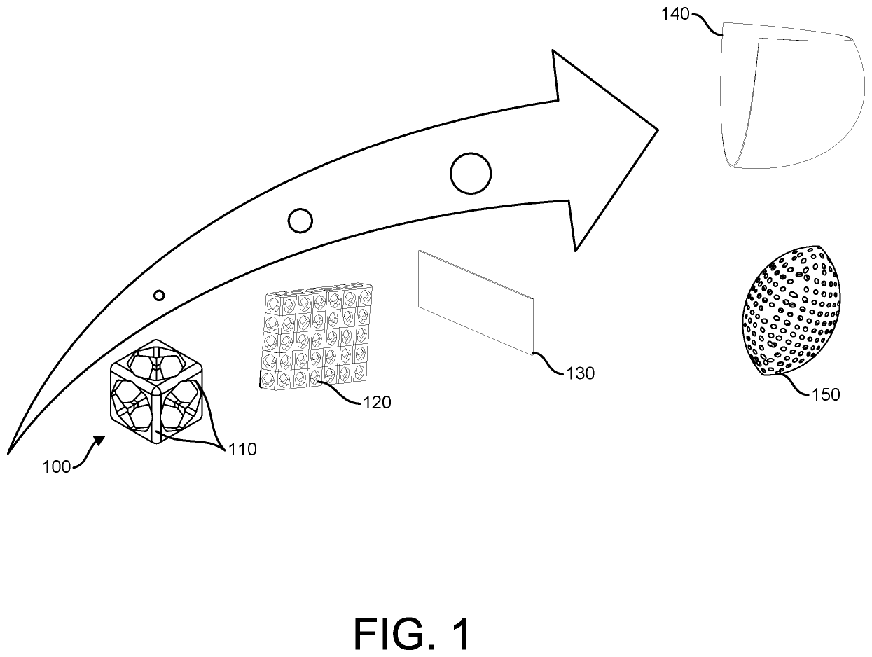

FIG. 1 is a diagram illustrating some example bioscaffold implants, in accordance with various embodiments. Bioscaffold implants can be constructed from bioscaffold structures having a base unit cell structure 100 having a given geometry. Each unit cell can comprise a plurality of filaments 110 composed of, for example, an extracellular material containing, for example, collagen I and collagen III.

The plurality of unit cells 100 (generally composed of a plurality of filaments) can be connected to form, for example, a monolayer bioscaffold structure 120. Other structures such as, for example, a bilayer or multilayer structure can also be contemplated. The plurality of connected unit cells in the bioscaffold structure can also be bioprinted, based on certain needs, to form, for example, a bioscaffold implant shaped as a substantially planar sheet or 3D macrostructure as discussed below.

In accordance with various embodiments, the bioscaffold implant can take many other forms including, for example, membranes, microbeads, fleece, fibers, gels and fiber meshes. A finished product mesh, for example, can provide the requisite porosity to allow optimal cellular infiltration and provide a large enough niche for cells to attach, and ultimately direct cell fate towards a remodeling/regenerative phenotype rather than fibrotic/contractile phenotype. Furthermore, from a mechanical/structural perspective, the arrangement of the unit cell and scaffold structure provides the appropriate mechanical strength and elasticity for the implant to be physiologically relevant as well as useful as a supportive matrix. These features can be provided by the bioscaffold structure using, for example, an optimal extracellular material composition, such as one containing Collagen I and III, thereby providing the necessary structural integrity properties. Determining the optimal composition to provide the requisite integrity can be accomplished by systemically varying, for example, pore size, angular filament deposition range, density, height, polymer type and filament size (e.g., diameter).

Returning to FIG. 1, in accordance with various embodiments, the final tissue construct of the bioscaffold implant can be, for example, a flat sheet 130 that is two-dimensional. As also provided by way of example in FIG. 1, another and more advanced final tissue construct of the bioscaffold implant can be, for example, molded sheets/constructs 140 that provide a hand in glove fit for tissue reinforcement, breast implants or other medical devices, anatomy or physiology within the breast (e.g., breast pocket), other organs, or other anatomy. As also provided by way of example in FIG. 1, yet another advanced final tissue construct of the bioscaffold implant can be, for example, a lumpectomy defect implant 150 within the breast (e.g., breast pocket), other organs, or other anatomy. The solid, implant-style construct can be printed, for example, from a range of from about 20 g to about 4500 g sizes for breast applications (e.g., lumpectomy) or custom sized for other anatomic applications. For example, the implant can be a prolate spheroid shaped (i.e. football) (as exemplary illustrated in FIG. 1), custom shaped, or shaped in another pre-determined geometrical configuration.

Such final tissue constructs of bioscaffold implants advantageously provide improved ease of use during the implant procedure by minimizing intraoperative manipulation while improve procedural efficiency for patients. For example, a three-dimensional (3D) construct, in accordance with various embodiments, would provide patients with a ‘ready-to-implant’ option, improving procedural efficiency for physicians and patients in the areas of, for example, tissue reinforcement and lumpectomy implants. By contrast, current cadaveric acellular dermal matrix (ADM) offerings are supplied as two-dimensional (2D) sheets of tissue, requiring extensive manipulation of the tissue sheet necessitating the surgeon to sew (or bind or connect) the acellular dermal matrix sheet into a pouch-like structure prior to implantation to create an adequate 3D pocket for a breast implant or tissue reinforcement application. This extensive manipulation requires additional procedure time and introduces numerous points of potential contamination.

Moreover, ADMs are hampered by tissue quality (age, smoking history, drug use, etc.), varied national regulatory constraints, donor availability, donor matching, host immune status, cost, and so on. By contrast, bioprinted scaffolds, in accordance with various embodiments herein, advantageously eliminates issues of donor availability, variability and quality/health status, tissue quality and regulatory policies. Moreover, by being bioprinted using controlled parameters (discussed in detail below) of the scaffold to meet required properties of the end-product bioscaffold implant, the bioprinted scaffolds allow for personalized medical applications that ADMs simply do not. These personalized medical applications can include, for example only and not limited to, lumpectomies (as discussed above), tubular cartilage applications, valvular heart disease applications, and coronary artery disease applications, hernia repair applications, tissue graft applications, venous, arterial and lymphatic vessel applications, structural applications or supportive applications where soft tissue defects exist.

Regarding tissue reinforcement, the uses and advantages of a biologic acellular dermal matrix, in accordance with various embodiments, are varied and substantial. One exemplary use is attachment to the inferior portion of the pectoralis muscle thus allowing for greater initial tissue expander fill volume for a two-stage breast reconstruction or implant placement for a single stage breast reconstruction. Another exemplary use is implant position maintenance (support) by helping to define the shape of the breast pocket by defining the inframammary fold, supporting the implant in a pre-pectoral breast reconstruction, and correcting implant malposition such as symmastia, bottoming out, etc. Another exemplary use is aesthetic defect camouflaging by using the scaffold as a buffer or a means to thicken tissue to mask unwanted cosmetic outcomes such as rippling. Other exemplary uses and benefits are capsular contracture reduction and more positive tissue response during radiation treatments.

Additionally, currently available tissue offerings may have inconsistent surface topography throughout. In the case of woven, spun or knitted synthetic constructs, the lack of appropriate microarchitecture affects the ability of host cells to recognize the graft as self, and promote cell adhesion, and thus inhibit a robust regenerative response. By controlling the combination of components in the scaffold to provide specific physical properties and implant outcomes, in accordance with various embodiments herein, the construct will have consistent surface topography throughout with an engineered microarchitecture (controlling microarchitecture properties such as, for example, porosity, fiber diameter, spacing, height of matrix, fiber orientation, etc.) that provides the appropriate scaffold for a robust wound healing, regenerative, infiltrative and remodeling response.

Moreover, the construct will provide, for example, cushioning and structural support for other tissues, supplemental support, protection, reinforcement and covering within the breast, other organs, or other anatomy and surrounding tissue, while stimulating host cell remodeling. The construct will allow, for example, plastic and reconstructive surgeons to support, repair, elevate and reinforce deficiencies where weakness or voids exist in, for example, the breast, other organs, or other anatomy and surrounding tissue that requires the addition of material to obtain the desired surgical outcome. Furthermore, the construct will allow for the repair of fascial defects within the breast, other organs, or other anatomy and surrounding tissues that require the addition of a reinforcing or bridging material to obtain a desired surgical result.

### Compositions

In one aspect, an acellular matrix bioscaffold composition is provided, the bioscaffold composition including a polymer, an extracellular matrix, and optionally other bioresorbable synthetic components. The unique combination of these components together provides the appropriate support matrix for remodeling. The combination of the polymeric and biologic construct materials also provides a robust scaffold for the addition of other molecular moieties. The extracellular matrix may comprise one or more of collagen (e.g., collagen 1 (Col-1), collagen III (Col-3), other types of collagen), extracellular matrix proteins (e.g., laminin, fibronectin, elastin, glycosaminoglycans, or combinations thereof), growth factors, cytokines, selected cell populations, small molecules, small molecule inhibitors, antibiotics, antimicrobials, nanoparticles, mesoporous silica, silk fibroin, and enzymatic degradation sites. The bioresorbable synthetic components may be added to the bioscaffold as needed to achieve the desired tensile strength and appropriate elastic modulus. High strength fibers may be added to the bioscaffold based upon the biomechanical and strength needs of the bioscaffold.

The natural or synthetic polymer in the bioscaffold composition may be any polymer that provides mechanical stability with a consistent degradation profile that allows plastic and reconstructive surgeons the ability to better predict and control outcomes in patients. The acellular matrix bioscaffold composition may comprise any suitable natural or synthetic polymer or combinations or blends thereof. The polymer can include, for example, poly(ethylene glycol) (PEG), poly(lactide-co-clycolide) (PLGA), polycaprolactone (PCL), poly(l-lactic acid) (PLLA), alginate, hyaluronic acid, gelatin, soy protein, fibrinogen, chitosan, dextran, starch, or any other type of polymer.

In various embodiments, the bioscaffold composition can contain Collagen I to Collagen III ratio similar to those contained within human dermis (e.g., fetal, adolescent, adult and elderly) as shown in Table I.

TABLE I

| Dermis Type | Collagen I/III Ratio |
|---|---|
| Fetus | 0.95 ± 0.03 |
| Adolescent | 2.27 ± 0.13 |
| Adult | 2.46 ± 0.15 |
| Elderly | 2.97 ± 0.40 |

That is, in various embodiments, the extracellular material can have a Collagen I to Collagen III ratio in the range of between about 0.5 to about 3.5, or in the range of between about 0.75 to about 3.0. More specific ratios of Collagen I to Collagen III can include about 0.5, about 0.55, about 0.6, about 0.65, about 0.7, about 0.75, about 0.8, about 0.85, about 0.9, about 0.95, about 1.0, about 1.05, about 1.1, about 1.15, about 1.2, about 1.25, about 1.3, about 1.35, about 1.4, about 1.45, about 1.5, about 1.55, about 1.6, about 1.65, about 1.7, about 1.75, about 1.8, about 1.85, about 1.9, about 1.95, about 2.0, about 2.05, about 2.1, about 2.15, about 2.2, about 2.25, about 2.3, about 2.35, about 2.4, about 2.45, about 2.5, about 2.55, about 2.6, about 2.65, about 2.7, about 2.75, about 2.8, about 2.85, about 2.9, about 2.95, about 3.0, about 3.05, about 3.1, about 3.15, about 3.2, about 3.25, about 3.3, about 2.35, about 3.4, about 3.45, about 3.5 and ranges between any two of these values.

In one aspect, the bioscaffold contains Col-1 and Col-3 base ratios similar to those contained within fetal and adolescent dermis. The ratio of Col-1 to Col-3 may be between about 0.90 to about 2.3, between about 0.95 to about 2.27 or between about 1 to about 2. In various embodiments, the ratio of Col-1 to Col-3 may be less than about 3, less than about 2.9, less than about 2.5 or less than about 2.46.

If desired, the bioscaffold composition may contain growth factors. The growth factors may be any one or combination of e.g., GM-CSF, NGF, SCF, TGF-β, EGF, VEGF and others.

The bioscaffold composition may further comprise cytokines. The cytokines may be any one or combination of e.g., IL-1, IL-4, IL-5, IL-6, IL-9, IL-13, IL-18, IL-25, IFN-α, IFN-β, and others.

If desired, the bioscaffold may further comprise antibiotics. Suitable antibiotics include a macrolide (e.g., azithromycin, clarithromycin and erythromycin), a tetracycline (e.g., doxycycline, tigecycline), a fluoroquinolone (e.g., gemifloxacin, levofloxacin, ciprofloxacin and mocifloxacin), a cephalosporin (e.g., ceftriaxone, defotaxime, ceftazidime, cefepime), a penicillin (e.g., amoxicillin, amoxicillin with clavulanate, ampicillin, piperacillin, and ticarcillin) optionally with a β-lactamase inhibitor (e.g., sulbactam, tazobactam and clavulanic acid), such as ampicillin-sulbactam, piperacillin-tazobactam and ticarcillin with clavulanate, an aminoglycoside (e.g., amikacin, arbekacin, gentamicin, kanamycin, neomycin, netilmicin, paromomycin, rhodostreptomycin, streptomycin, tobramycin, and apramycin), a penem or carbapenem (e.g. doripenem, ertapenem, imipenem and meropenem), a monobactam (e.g., aztreonam), an oxazolidinone (e.g., linezolid), vancomycin, glycopeptide antibiotics (e.g. telavancin), tuberculosis-mycobacterium antibiotics and the like.

The bioscaffold composition may further comprise antimicrobials, including antibacterials, antifungals (e.g., polyene antifungals, such as amphotericin B; triazole antifungals, such as itraconazole, ketoconazole, fluconazole, voriconazole, clotrimazole, Isavuconazole, miconazole and posaconazole; echinocandin antifungals, such as caspofungin, micafungin, and anidulafungin, orotomide antifungals, such as F901318, which inhibits dihydroorotate dehydrogenase), antivirals (e.g., oseltamivir, zanamavir, amantidine, rimantadine, ribavirin, gancyclovir, valgancyclovir, foscavir, Cytogam® (Cytomegalovirus Immune Globulin), pleconaril, rupintrivir, palivizumab, motavizumab, cytarabine, docosanol, denotivir, cidofovir, and acyclovir), antiparasitics or combinations thereof.

In one aspect, an acellular bioscaffold composition is provided, the bioscaffold composition including 1) a polymer, 2) collagen I (Col-1), and 3) collagen III (Col-3), wherein the ratio of collagen I to collagen III is between about 0.5 and about 3.5. In various embodiments, the ratio can be between about 0.9 and about 2.3. In various embodiments, the polymer may be polycaprolactone (PCL), or a blend containing PCL, and the ratio of Col-1 to Col-3 is about 1. The polymer can be a blend of polymers. The polymer can be a blend containing PCL. In accordance with various embodiments, the polymer can be a blend of polymers.

In another aspect, an acellular matrix bioscaffold composition is provided, the composition comprising: 1) a natural or synthetic polymer; 2) collagen I (Col-1); 3) collagen III (Col-3), and 4) polycaprolactone (PCL), wherein said bioscaffold is synthetic and the ratio of Col-1 to Col-3 is between 0.5 and 3.5.

In another aspect, an acellular bioscaffold composition is provided, the bioscaffold composition including 1) a polymer, 2) collagen I (Col-1), and 3) collagen III (Col-3), wherein the ratio of Col-1 to Col-3 is between about 0.9 and about 2.3, with the proviso that the bioscaffold does not comprise the bio-active molecules present in cadaveric acellular dermal matrix (ADM). The acellular bioscaffold composition, in accordance with various embodiments, will lead to a diminished immunogenic response compared to ADM, due to the lack of bio-active molecules present in cadaveric ADM. The polymer can be a blend of polymers.

The bioscaffold composition may be porous and have a microstructure comprising pore diameters of about 50-1000 microns, about 100-900 microns, about 200-800 microns, about 300-700 microns, about 400-600 microns, about 100-500 microns, about 200-400 microns, about 150-300 microns, about 200-250 microns, about 100 microns, about 150 microns, about 200 microns, about 250 microns, about 300 microns, about 350 microns, about 400 microns, about 450 microns, about 500 microns, and ranges between any two of these values. The size of the pore is sufficient to allow for appropriate host cell ingrowth, attachment, and/or incorporation. The bioscaffold can also provide the appropriate microstructure for cellular ingrowth and vascularization, while providing similar support and biomechanical properties to the resected tissue and/or extracellular matrix. Alternatively, the plurality of pores can be a shape other than circular, wherein the at least one opening can have a length of between about 100 microns to about 500 microns, or about 50 microns to about 1000 microns, or between about 50 microns to about 500 microns, or about 100 microns to about 1000 microns. Each fiber (fibril) of the bioscaffold structure can have a diameter of less than or equal to about 100 microns.

The bioscaffold composition may have a macrostructure that is either small, medium, or large pre-printed implant that is pre-manufactured for implantation during the time of the index procedure. In another aspect, the bioscaffold may have a macrostructure that is customized and patient specific, printed following the index procedure at the surgeon's discretion/direction.

Degradation of the bioscaffold composition is expected over time following surgical implantation. It is intended that host cell integration into the porous bioscaffold will proceed throughout the degradation process, such that infiltrating cells will degrade the polymer and secrete their own extracellular matrix in an effort to regenerate that tissue. The bioscaffold composition degradation profile is defined and predictable across manufactured lots, which is an improvement over the currently available acellular dermal matrix constructs.

In one aspect, the bioscaffold composition may further comprise pro-angiogenic (e.g., VEGF, PlGF) bioactive molecules to promote vascularization in patients with compromised vascularity.

In one aspect, the bioscaffold composition may further comprise anti-fibrotic molecules (anti-TGFβ, anti-TNF-α) to reduce fibrosis in patients following radiation.

In one aspect, the bioscaffold composition may further comprise factors affecting adipogenesis and proliferation (e.g., IGF-1, Dexamethasone) to promote grafted adipose cell growth.

### Method of Making Bioscaffold

The bioscaffold compositions discussed above can be made into 2D and 3D shapes (that can support tissue and organ growth) using a variety of different additive manufacturing techniques, which are processes involving the use of digital 3D design data to build up an extracellular construct in layers by depositing successive layers of the bioscaffold compositions described herein. Additive manufacturing processes can include, for example, material jetting (or ink-jet) 3D printing, extrusion 3D printing, fused deposition 3D printing, liquid material 3D printing, 3D melt electrowriting, 3D melt electrospinning and so on. Additive manufacturing processes offer some unique advantages over other extracellular component manufacturing techniques with respect to the manufacture of porous three-dimensional extracellular structures due to the complexities of the geometries and structural elements of the unit cells that comprise those types of structures. For example, additive manufacturing provides the advantage that specific bioactive components or ECM molecules can be placed in certain locations within the construct to spatially direct remodeling.

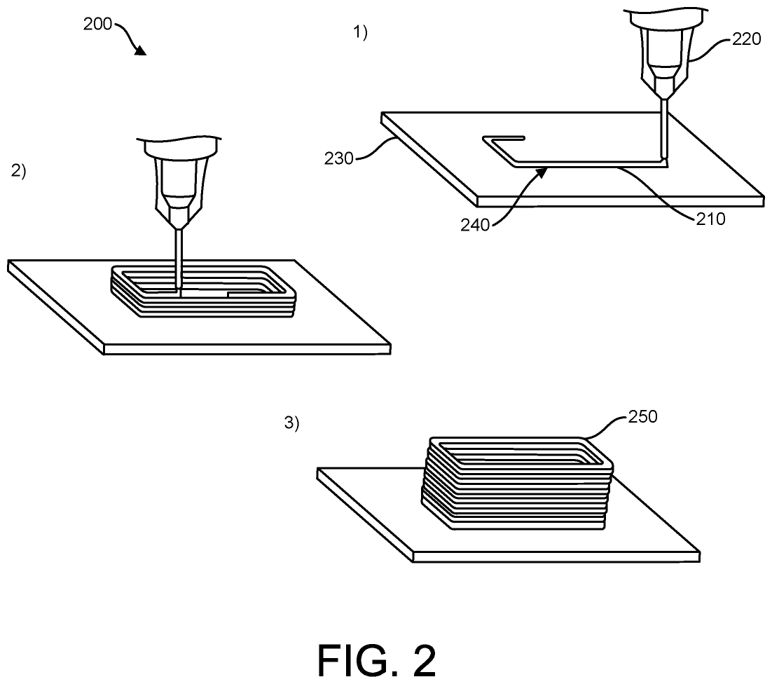

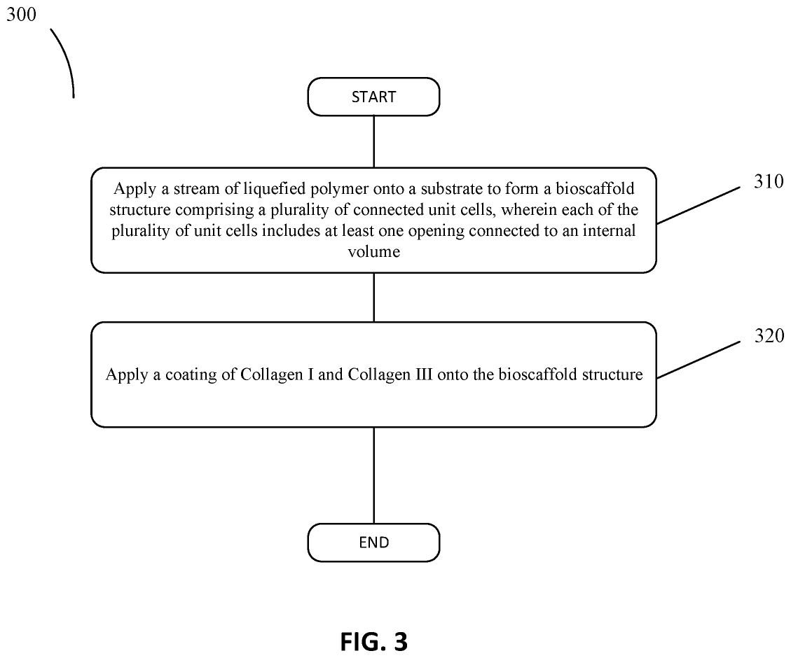

In accordance with various embodiments, a method 200 for producing a bioscaffold structure is provided, as illustrated in FIG. 2, and provided for by method 300 of FIG. 3. As provided in FIG. 3, method 300 can comprise, at step 310, applying a stream of liquefied polymer onto a substrate form a bioscaffold structure comprising a plurality of connected unit cells, wherein each of the plurality of unit cells includes at least one opening connected to an internal volume. In various embodiments, the liquefied polymer is comprised of a solution or mixture containing one or more polymer components. In various embodiments, the liquefied polymer is comprised of a polymer material that has been heated beyond its melting point to a fluid state.

In various embodiments, step 310 can be repeated such that the streams of liquefied polymer are successively deposited on top of each other to form the bioscaffold structure.

Method 300 further comprises, at step 320, applying a (e.g., Collagen I, Collagen II, Collagen III, Collagen IV, Collagen V, etc.) coating onto and/or infusion into the bioscaffold structure.

In various embodiments, the collagen coating and/or infusion includes Collagen I and Collagen III. In various embodiments, the collagen coating can contain a Collagen I to Collagen III ratio similar to those contained within fetal and adolescent dermis. Conventional tissue offerings, which are predominantly taken from adult and elderly donors, contain a higher ratio (i.e., greater than about 2.4) of Collagen I to Collagen III, thereby providing decreased support, cushioning, protection, reinforcement and covering than do tissues containing lower ratios.

In various embodiments, the collagen coating can have a Collagen I to Collagen III ratio in the range of between about 0.5 to about 3.5, or in the range of between about 0.75 to about 3.0, or in the preferred range of between about 0.9 to about 2.5. The preferred Collagen I to Collagen III ratio range of between about 0.9 to about 2.5 may offer advantages against conventional tissue offerings, which are predominantly taken from adult and elderly donors, contain much higher ratios of Collagen I to Collagen III, thereby providing decreased support, cushioning, protection, reinforcement and covering than do tissues containing lower ratios.

More specific ratios of Collagen I to Collagen III can include about 0.5, about 0.55, about 0.6, about 0.65, about 0.7, about 0.75, about 0.8, about 0.85, about 0.9, about 0.95, about 1.0, about 1.05, about 1.1, about 1.15, about 1.2, about 1.25, about 1.3, about 1.35, about 1.4, about 1.45, about 1.5, about 1.55, about 1.6, about 1.65, about 1.7, about 1.75, about 1.8, about 1.85, about 1.9, about 1.95, about 2.0, about 2.05, about 2.1, about 2.15, about 2.2, about 2.25, about 2.3, about 2.35, about 2.4, about 2.45, about 2.5, about 2.55, about 2.6, about 2.65, about 2.7, about 2.75, about 2.8, about 2.85, about 2.9, about 2.95, about 3.0, about 3.05, about 3.1, about 3.15, about 3.2, about 3.25, about 3.3, about 2.35, about 3.4, about 3.45, about 3.5 and ranges between any two of these values.

In various embodiments, the collagen (e.g., Collagen I, Collagen II, Collagen III, Collagen IV, Collagen V, etc.) and/or other ECM materials can be combined with the liquefied polymer to form a bioink that can be deposited to form the bioscaffold structure in a single process step instead of successive process steps.

In various embodiments, the collagen and/or ECM material coating can be lypholized to sublime the solvent used to create the suspension thus allowing the collagen and/or ECM material coating to adhere to the bioscaffold structure.

In various embodiments, the bioscaffold structure can be scanned with an electron beam, UV light or chemical agent such as EDAC, a carbodiimide crosslinker, or other physical or chemical crosslinker following application of the collagen or ECM coating in order to crosslink the polymers and increase the structural strength and integrity of the bioscaffold structure.

In various embodiments, the at least one opening (e.g., pore) of each of the plurality of unit cells can have a diameter of about 50-1000 microns, about 100-900 microns, about 200-800 microns, about 300-700 microns, about 400-600 microns, about 100-500 microns, about 200-400 microns, about 150-300 microns, about 200-250 microns, about 100 microns, about 150 microns, about 200 microns, about 250 microns, about 300 microns, about 350 microns, about 400 microns, about 450 microns, about 500 microns, and ranges between any two of these values. Moreover, each of the plurality of unit cells can comprise filaments that have a diameter of less than or equal to about 100 microns. The plurality of connected unit cells can also form a substantially planar sheet. Further, the plurality of connected unit cells can form a 3D macrostructure. Further, the bioscaffold structure can have a thickness of between about 0.5 mm to about 200 mm, or between about 0.5 mm to about 20 mm, or between about 0.5 mm to about 2.5 mm. The 3D macrostructure can include, for example, a “hand in glove” fit configuration and a “ready-to-use” solid implant configuration discussed previously.

Alternatively, the plurality of pores can be a shape other than circular, wherein the at least one opening can have a length of between about 100 microns to about 500 microns, or about 50 microns to about 1000 microns, or between about 50 microns to about 500 microns, or about 100 microns to about 1000 microns. Each fiber (fibril) of the bioscaffold structure can have a diameter of less than or equal to about 100 microns.

As illustrated in FIG. 2, step (1), a stream of liquefied polymer 210 can be deposited, via syringe or nozzle (or any other stream deposition device) 220, onto a substrate 230. The liquid polymer stream 210 can form a single layer 240. As shown in step (2), the depositing process can be repeated to add additional liquefied polymer layers to increase the thickness of the liquefied polymer deposition until a desired thickness is achieved for a finished product 250, as provided in step (3). The liquefied polymer streams can form a single or multiple layer(s) comprised of a plurality of unit cell structures. The finished product can be, as discussed above, a substantially planar sheet or a 3D macrostructure.

Each unit cell of the bioscaffold structure can comprise a polymer, or blend of polymers with a collagen and/or ECM material coating. The polymer can be polycaprolactone (PCL). The composition that is deposited can be in the form of a slurry of polymer with the Collagen I/Collagen III embedded, mixed, or encapsulated with the polymer. Alternatively, a coating of Collagen I/Collagen III and streams of liquefied polymer can be deposited separately and then interlaced to form the structure. The deposition can be performed, for example, by a bioprinter using components such as, for example, a nozzle or syringe. These components can be, for example, pneumatically, piston, or screw driven. Pneumatic driven syringes, for example, can deposit liquefied polymer in sequential layers to create the construct, which will ultimately be cross-linked. Bioactive molecules (e.g., Collagen I/Collagen III based bioinks with any other additional bioactive components as discussed above) can either be co-printed along with the base polymer or layered upon the base polymer layer sequentially. The polymer can be a blend of polymers. The bioscaffold can comprise an ECM material coating instead of a collagen coating, or a combined collagen/ECM material coating.

As compared to traditional polymer-based compositions, which can be deposited via additive manufacturing techniques under heated conditions and relatively higher pressures to promote scaffold formation while still maintaining structural stability of the polymer-based composition, certain bioinks containing bioactive molecules in addition to polymers may have to be deposited under milder conditions relative to polymer-based compositions. This can be due to the relatively more delicate nature of bioink structures (e.g., higher water content, non-crystalline structure, etc.). As such, bioprinting process parameters such as printing pressure or nozzle/syringe diameter can be considered in reducing the shear stress on some bioinks to prevent damaged or lysed cells, which can affect cell viability in the bioinks. Other parameters that may be considered, and correspondingly controlled include, for example, printing temperature (e.g., lower temperature than polymer-based compositions), uniformity in diameter of the filaments that make up the unit cell, angles at the interaction of filaments, bleeding of filaments together at intersects, and maintenance of shape fidelity after printing but before cross-linking with polymer-based compositions. Therefore, and in accordance with various embodiments, the polymer and bioactive molecules (e.g., Collagen I/Collagen III and any other additional bioactive components as discussed above) can be deposited as, for example, droplets or streams (see below for more detail) separately utilizing separately defined process parameters to ensure scaffold manufacturing while maintaining structural integrity of each respective deposited compositions. The deposition of the polymers and bioactive molecules (e.g., Collagen I/Collagen III and any other additional bioactive components as discussed above) can occur, as stated above, separately and then interlaced or cross-linked to form the scaffold. To accomplish this, the bioactive molecules can either be co-printed along with the base polymer or layered upon the base polymer layer sequentially.

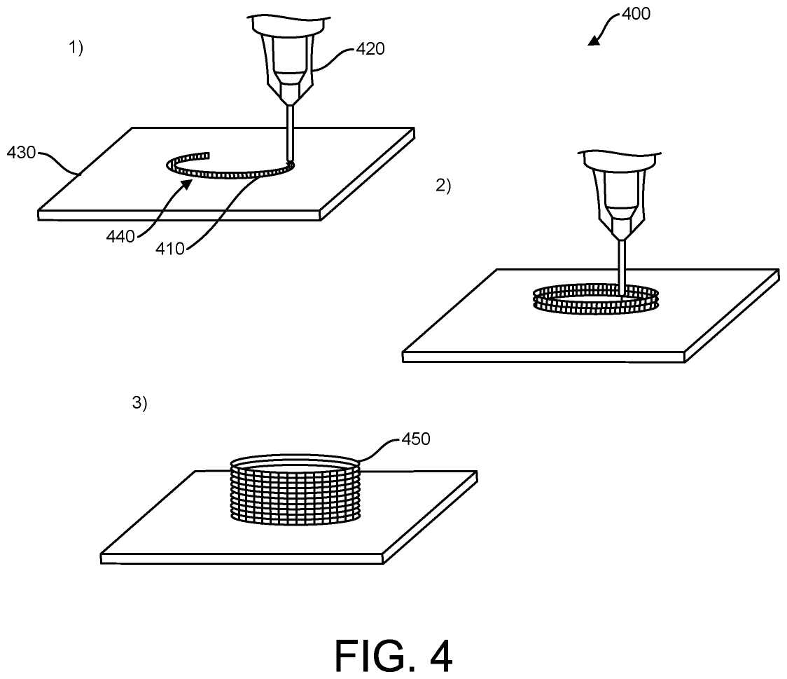

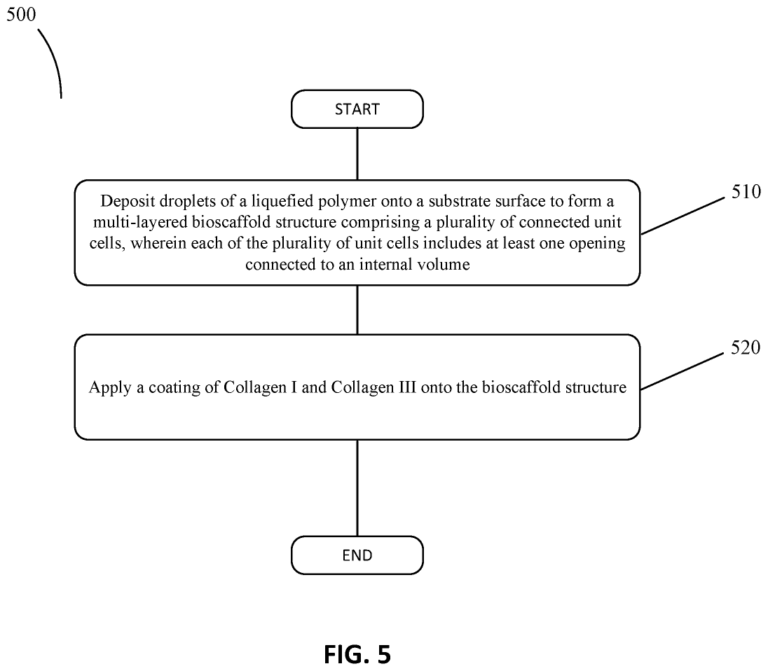

In accordance with various embodiments, a method 400 for producing a bioscaffold structure is provided, as illustrated in FIG. 4, and provided by method 500 of FIG. 5. As provided in FIG. 5, method 500 can comprise, at step 510, depositing droplets of a liquefied polymer onto a substrate surface to form a bioscaffold structure comprising a plurality of connected unit cells, wherein each of the plurality of unit cells includes at least one opening connected to an internal volume. In various embodiments, the liquefied polymer is comprised of a solution or mixture containing one or more polymer components. In various embodiments, the liquefied polymer is comprised of a polymer material that has been heated beyond its melting point to a fluid state.

In various embodiments, step 510 can be repeated such that the drops of liquefied polymer are successively deposited on top of each other to form the bioscaffold structure.

Method 500 further comprises, at step 520, applying a (e.g., Collagen I, Collagen II, Collagen III, Collagen IV, Collagen V, etc.) coating onto the bioscaffold structure.

In various embodiments, the collagen coating includes Collagen I and Collagen III. In various embodiments, the collagen coating can contain a Collagen I to Collagen III ratio similar to those contained within fetal and adolescent dermis. Current tissue offerings, which are predominantly taken from adult and elderly donors, contain much higher ratios of Collagen I to Collagen III, thereby providing decreased support, cushioning, protection, reinforcement and covering than do tissues containing lower ratios.

In various embodiments, the collagen coating can have a Collagen I to Collagen III ratio in the range of between about 0.5 to about 3.5, or in the range of between about 0.75 to about 3.0, or in the preferred range of between about 0.9 to about 2.5. The preferred Collagen I to Collagen III ratio range of between about 0.9 to about 2.5 may offer advantages against conventional tissue offerings, which are predominantly taken from adult and elderly donors, contain much higher ratios of Collagen I to Collagen III, thereby providing decreased support, cushioning, protection, reinforcement and covering than do tissues containing lower ratios.

More specific ratios of Collagen I to Collagen III can include about 0.5, about 0.55, about 0.6, about 0.65, about 0.7, about 0.75, about 0.8, about 0.85, about 0.9, about 0.95, about 1.0, about 1.05, about 1.1, about 1.15, about 1.2, about 1.25, about 1.3, about 1.35, about 1.4, about 1.45, about 1.5, about 1.55, about 1.6, about 1.65, about 1.7, about 1.75, about 1.8, about 1.85, about 1.9, about 1.95, about 2.0, about 2.05, about 2.1, about 2.15, about 2.2, about 2.25, about 2.3, about 2.35, about 2.4, about 2.45, about 2.5, about 2.55, about 2.6, about 2.65, about 2.7, about 2.75, about 2.8, about 2.85, about 2.9, about 2.95, about 3.0, about 3.05, about 3.1, about 3.15, about 3.2, about 3.25, about 3.3, about 2.35, about 3.4, about 3.45, about 3.5 and ranges between any two of these values.

In various embodiments, the collagen (e.g., Collagen I, Collagen II, Collagen III, Collagen IV, Collagen V, etc.) and/or other ECM materials can be combined with the liquefied polymer to form a bioink that can be deposited to form the bioscaffold structure in a single process step instead of successive process steps.

In various embodiments, the collagen and/or ECM material coating can be lypholized to sublime the solvent used to create the suspension thus allowing the collagen and/or ECM material coating to adhere to the bioscaffold structure.

In various embodiments, the bioscaffold structure can be scanned with an electron beam, UV light or chemical agent such as EDAC, a carbodiimide crosslinker, or other physical or chemical crosslinker following application of the collagen and/or ECM material coating in order to crosslink the polymers and increase the structural strength and integrity of the bioscaffold structure.

In various embodiments, the at least one opening (e.g., pore) of each of the plurality of unit cells can have a diameter of about 50-1000 microns, about 100-900 microns, about 200-800 microns, about 300-700 microns, about 400-600 microns, about 100-500 microns, about 200-400 microns, about 150-300 microns, about 200-250 microns, about 100 microns, about 150 microns, about 200 microns, about 250 microns, about 300 microns, about 350 microns, about 400 microns, about 450 microns, about 500 microns, and ranges between any two of these values. Moreover, each of the plurality of unit cells can comprise filaments that have a diameter of less than or equal to about 100 microns. The plurality of connected unit cells can also form a substantially planar sheet. Further, the plurality of connected unit cells can form a 3D macrostructure. Further, the bioscaffold structure can have a thickness of between about 0.5 mm to about 200 mm, or between about 0.5 mm to about 20 mm, or between about 0.5 mm to about 2.5 mm. The 3D macrostructure can include, for example, a “hand in glove” fit configuration and a “ready-to-use” solid implant configuration discussed previously.

As illustrated in FIG. 4, step (1), liquefied polymer droplets 410 can be deposited, via syringe or nozzle (or any other deposition device) 420, onto a substrate 430. Droplets 410 can form a single layer 440. As shown in step (2), the depositing process can be repeated to add additional liquefied polymer layers to increase the thickness of the liquefied polymer deposition until a desired thickness is achieved for a finished product 450, as provided in step (3). The droplets of liquefied polymer can form a single or multiple layer(s) comprised of a plurality of unit cell structures. The finished product can be, as discussed above, a substantially planar sheet or a 3D macrostructure. The 3D macrostructure can include, for example, a “hand in glove” fit configuration and a “ready-to-use” solid implant configuration discussed in more detail above.

Each unit cell of the bioscaffold structure can comprise a polymer, or blend of polymers, with a collagen and/or ECM material coating. The polymer can be polycaprolactone (PCL). The composition that is deposited can be in the form of a slurry of polymer with the Collagen I/Collagen III embedded, mixed, or encapsulated with the polymer. Alternatively, the droplets of Collagen I/Collagen III and droplets of liquefied polymer can be deposited separately and then interlaced to form the structure. The deposition can be performed, for example, by a bioprinter using components such as, for example, a nozzle or syringe. These components can be, for example, pneumatically, piston, or screw driven. Pneumatic driven syringes, for example, can deposit liquefied polymer in sequential layers to create the construct, which will ultimately be cross-linked. Bioactive molecules (e.g., Collagen I/Collagen III based bioinks with any other additional bioactive components as discussed above) can either be co-printed along with the base polymer or layered upon the base polymer layer sequentially. The polymer can be a blend of polymers. The bioscaffold can comprise an ECM material coating instead of a collagen coating, or a combination collagen/ECM coating.

As compared to traditional polymer-based compositions, which can be deposited via additive manufacturing techniques under heated conditions and relatively higher pressures to promote scaffold formation while still maintaining structural stability of the polymer-based composition, certain bioinks containing bioactive molecules in addition to polymer may have to be deposited under milder conditions relative to polymer-based compositions. This can be due to the relatively more delicate nature of bioink structures (e.g., higher water content, non-crystalline structure, etc.). As such, bioprinting process parameters such as printing pressure or nozzle/syringe diameter can be considered in reducing the shear stress on some bioinks to prevent damaged or lysed cells, which can affect cell viability in the bioinks. Other parameters that may be considered, and correspondingly controlled include, for example, printing temperature (e.g., lower temperature than polymer-based compositions), uniformity in diameter of the filaments that make up the unit cell, angles at the interaction of filaments, bleeding of filaments together at intersects, and maintenance of shape fidelity after printing but before cross-linking with polymer-based compositions. Therefore, and in accordance with various embodiments, the polymer and bioactive molecules (e.g., Collagen I/Collagen III and any other additional bioactive components as discussed above) can be deposited as, for example, droplets or streams (see below for more detail) separately utilizing separately defined process parameters to ensure scaffold manufacturing while maintaining structural integrity of each respective deposited compositions. The deposition of the polymers and bioactive molecules (e.g., Collagen I/Collagen III and any other additional bioactive components as discussed above) can occur, as stated above, separately and then interlaced or cross-linked to form the scaffold. To accomplish this, the bioactive molecules can either be co-printed along with the base polymer or layered upon the base polymer layer sequentially.

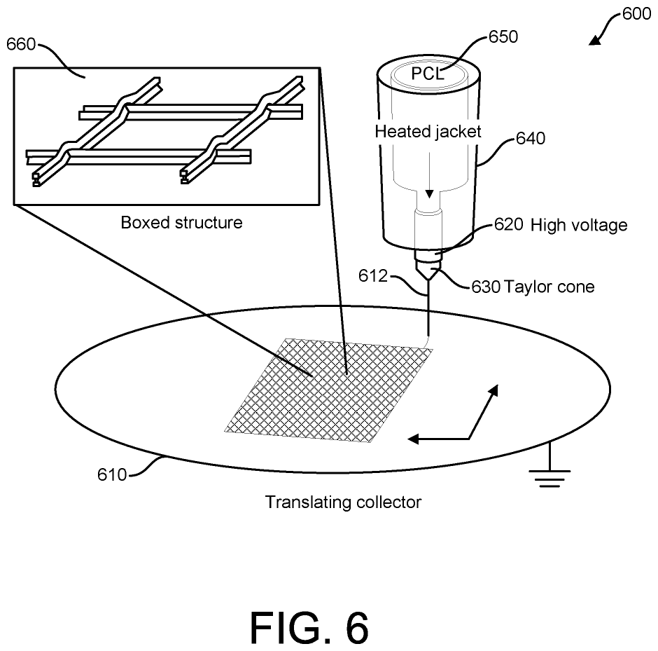

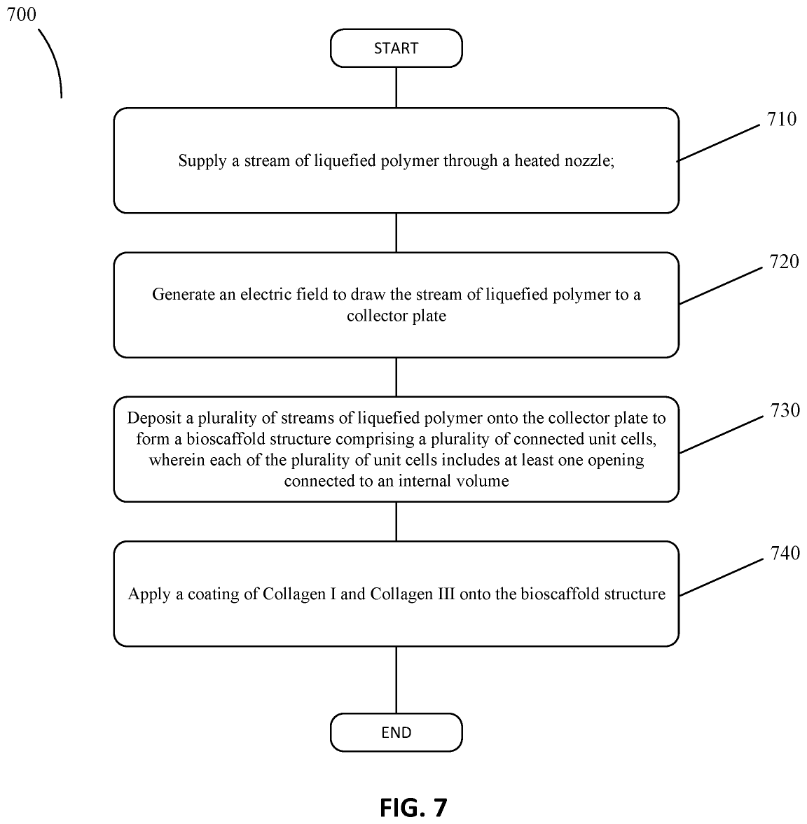

In accordance with various embodiments, a method 600 for producing a bioscaffold structure is provided as illustrated, for example, in FIG. 6, by method 700 of FIG. 7. As provided in FIG. 7, method 700 can comprise, at step 710, supplying a stream of liquefied polymer (e.g., PCL) through a heated nozzle. In various embodiments, the liquefied polymer is comprised of a solution or mixture containing one or more polymer components. In various embodiments, the liquefied polymer is comprised of a polymer material that has been heated beyond its melting point to a fluid state. Method 700 can further comprise, at step 720, generating an electric field to draw the stream of liquefied polymer to a collector plate. In various embodiments, the electric field is created by generating a high potential difference (voltage) between the nozzle head (spinneret) supplying the liquefied polymer and the collector plate. In step 730, a plurality of streams of liquefied polymer is deposited onto the collector plate to form a bioscaffold structure comprising a plurality of connected unit cells, wherein each of the plurality of unit cells includes at least one opening connected to an internal volume. In various embodiments, steps 710 and 720 are repeated such that a plurality of streams of liquefied polymer are successively deposited on top of each other to form the bioscaffold structure. In various embodiments, the strength of the electric field (and thus the voltage applied) generated is increased with each successive layer of polymer that is deposited and/or with the thickness of the bioscaffold structure.

In step 740, a coating of collagen (e.g., Collagen I, Collagen II, Collagen III, Collagen IV, Collagen V, etc.) is applied onto the bioscaffold. In various embodiments, the applied collagen coating is deposited on the external surfaces (i.e., outside surfaces) and internal surfaces (e.g., pores, pore volume surfaces, etc.) of the bioscaffold structure.

In various embodiments, the collagen coating includes Collagen I and Collagen III. In various embodiments, the collagen coating can contain a Collagen I to Collagen III ratio similar to that contained within human fetal and adolescent dermis. Conventional tissue offerings, which are predominantly taken from adult and elderly donors, contain a higher ratio (i.e., greater than about 2.4) of Collagen I to Collagen III, thereby providing decreased support, cushioning, protection, reinforcement and covering than do tissues containing lower ratios.

In various embodiments, the collagen coating can have a Collagen I to Collagen III ratio in the range of between about 0.5 to about 3.5, or in the range of between about 0.75 to about 3.0, or in the preferred range of between about 0.9 to about 2.5. The preferred Collagen I to Collagen III ratio range of between about 0.9 to about 2.5 may offer advantages against conventional tissue offerings, which are predominantly taken from adult and elderly donors, contain much higher ratios of Collagen I to Collagen III, thereby providing decreased support, cushioning, protection, reinforcement and covering than do tissues containing lower ratios.

More specific ratios of Collagen I to Collagen III can include about 0.5, about 0.55, about 0.6, about 0.65, about 0.7, about 0.75, about 0.8, about 0.85, about 0.9, about 0.95, about 1.0, about 1.05, about 1.1, about 1.15, about 1.2, about 1.25, about 1.3, about 1.35, about 1.4, about 1.45, about 1.5, about 1.55, about 1.6, about 1.65, about 1.7, about 1.75, about 1.8, about 1.85, about 1.9, about 1.95, about 2.0, about 2.05, about 2.1, about 2.15, about 2.2, about 2.25, about 2.3, about 2.35, about 2.4, about 2.45, about 2.5, about 2.55, about 2.6, about 2.65, about 2.7, about 2.75, about 2.8, about 2.85, about 2.9, about 2.95, about 3.0, about 3.05, about 3.1, about 3.15, about 3.2, about 3.25, about 3.3, about 2.35, about 3.4, about 3.45, about 3.5 and ranges between any two of these values.

In various embodiments, the collagen (e.g., Collagen I, Collagen II, Collagen III, Collagen IV, Collagen V, etc.) and/or other ECM materials can be combined with the liquefied polymer to form a bioink that can be deposited to form the bioscaffold structure in a single process step instead of successive process steps.

In various embodiments, the collagen and/or ECM material coating can be lypholized to sublime the solvent used to create the suspension thus allowing the collagen and/or ECM material coating to adhere to the bioscaffold structure.

In various embodiments, the bioscaffold structure can be scanned with an electron beam, UV light or chemical agent such as EDAC, a carbodiimide crosslinker, or other physical or chemical crosslinker following application of the collagen or ECM coating in order to crosslink the polymers and increase the structural strength and integrity of the bioscaffold structure.

As illustrated in FIG. 6, a stream of liquefied polymer 612 can be deposited, via a nozzle (spinneret) 620, onto a collector plate 610. A heating element (jacket) 640 can be used to raise the temperature of a solid or semi-solid polymer material 650 (for example, PCL) beyond its melting point to a fluid state to form a liquefied polymer. A stream of liquefied polymer can be drawn out of the nozzle 630 by an electric field generated by a high voltage element 620. The stream of liquefied polymer 612 can form a single or multiple layer(s) comprised of a plurality of unit cell structures 660. The depositing process can be repeated to add additional layers to increase the thickness of the liquefied polymer deposition until a desired thickness is achieved for a finished product. The finished product can be, as discussed above, a substantially planar sheet or a 3D macrostructure. The 3D macrostructure can include, for example, a “hand in glove” fit configuration and a “ready-to-use” solid implant configuration discussed in more detail above.

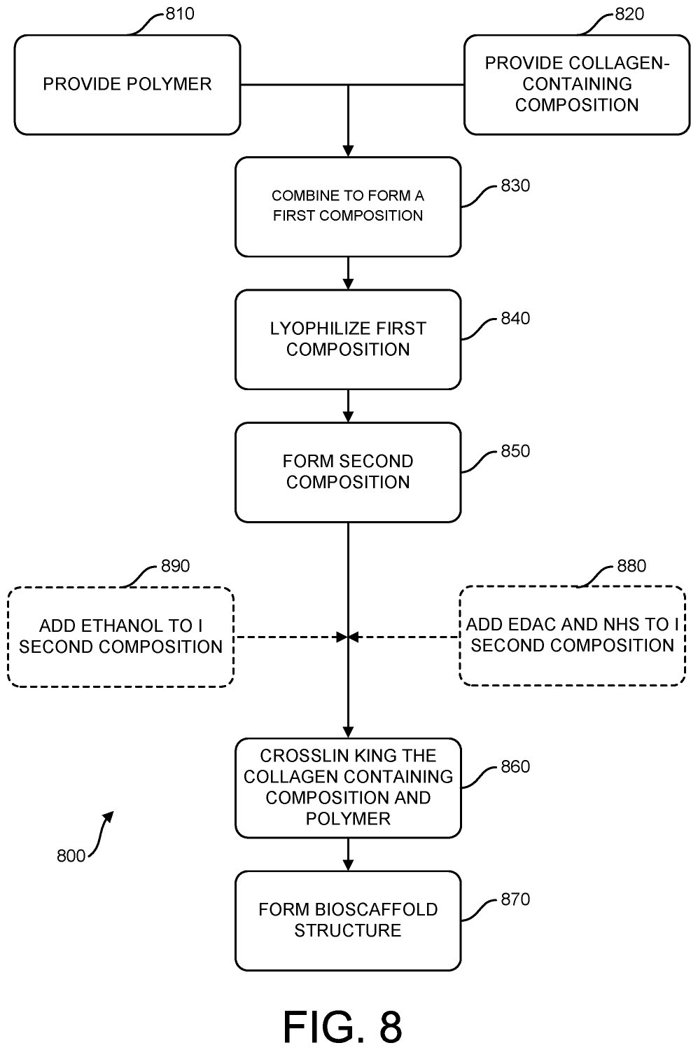

In accordance with various embodiments, a method for producing bioscaffold structures is provided as provided, for example, by method 800 of FIG. 8. As provided in FIG. 8, method 800 can comprise, providing a polymer at step 810, providing a collagen-containing composition at step 820, and combining, at step 830, the polymer and collagen containing composition to form a first composition. Various methods can be used for combining the polymer and collagen containing composition to form a first composition. For example, a solidified polymer construct could be dipped into the collagen containing composition, which can be provided in the form of a solution. The collagen containing solution could be coated or sprayed onto the polymer. In another example, for a polymer provided as a mesh (discussed below), the polymer mesh can be placed in a mold, which is then filled with collagen containing composition (in the form of a solution) at an amount to ensure all pores within the mesh were filled to ensure homogeneous coverage of collagen-containing composition across the mesh.

The method can further comprise, at step 840, lyophilizing the first composition to form, at step 850, a second composition. Various processing parameters can be used to ensure sufficient lyophilization. An example lyophilization process can include freezing the first composition down to about −40° C. at a controlled rate of about one degree per minute. This temperature can be held for about one hour to ensure all solvent in the solution freezes. The temperature can then be brought up to about −10° C. at a controlled rate of about one degree per minute and a vacuum is applied at approximately 0.2 millibarr. To help ensure evaporation of formed ice crystals, the −10° C. temperature and approximately 0.2 millibarr pressure can held for about 18 hours. The temperature can then be raised to about 20° C. at a controlled rate of one degree per minute, still under vacuum. This can be held indefinitely (e.g., for a minimum of 1 hour) to ensure the construct has dried.

The method can further comprise, at step 860, crosslinking the collagen-containing composition and the polymer in the second composition to, at step 870, form the bioscaffold structure.

In various embodiments, the collagen-containing composition of method 800 of FIG. 8 can include collagen I (Col-1), Collagen III (Col-3), or both Col-1 and Col-3. In various embodiments, the collagen-containing composition can include Col-1 and Col-3. In various embodiments, the collagen-containing composition can include Col-1 and Col-3, wherein the ratio of Col-1 to Col-3 can be between, for example, about 0.5 and about 3.5, or between about 0.9 and about 2.3. In various embodiments, the collagen-containing composition can include Col-1 and Col-3, wherein the ratio of Col-1 to Col-3 is about 1.

In various embodiments, the collagen-containing composition can include extracellular matrix proteins (ECMs) selected from the group consisting of laminin, fibronectin, elastin, glycosaminoglycans, and combinations thereof. In various embodiments, the collagen-containing composition can include elastin.

In various embodiments, the polymer can be selected from the group consisting of poly(ethylene glycol) (PEG), poly(lactide-co-clycolide) (PLGA), polycaprolactone (PCL), poly(l-lactic acid) (PLLA), alginate, hyaluronic acid, gelatin, soy protein, fibrinogen, chitosan, dextran, and starch. In various embodiments, the polymer is PCL. In various embodiments, the polymer is a blend of polymers. In various embodiments, the polymer can be provided as a polymer mesh. In various embodiments, the polymer can be provided as a PCL mesh. This mesh structure can be produced by various methods including, for example, various 3D printing methods such as those, for example, discussed herein.

In various embodiments, method 800 can further comprising crosslinking the collagen-containing composition and the polymer in the second composition with 1-Ethyl-3-(3-dimethylaminopropyl)carbodiimide (EDAC) to form the bioscaffold structure. EDAC is a water-soluble carbodiimide. EDAC can be generally utilized in the about 4.0 to about 6.0 pH range. EDAC can be used, for example, as a carboxyl activating agent for the coupling of primary amines to yield amide bonds, or to activate phosphate groups in order to form phosphomono-esters and phosphodiesters. Further example uses include peptide synthesis, protein crosslinking to nucleic acids, and preparation of immunoconjugates. Crosslinking can be accomplished various ways besides EDAC-based crosslinking. Examples of crosslinking promoters include, but are not limited to, ultraviolet irradiation, electron beam, gamma radiation, peroxide, vinylsilane, calcium chloride (CaCl2), glucose, glutaraldehyde, drying, heating, and various other chemical or physical methods.

Referring back to FIG. 8, in various embodiments, method 800 can further comprise crosslinking the collagen-containing composition and the polymer in the second composition with EDAC and N-hydroxysuccinimide (NHS), at step 880, to form the bioscaffold structure. NHS is an organic compound commonly used, in organic chemistry or biochemistry industries, as an activating reagent for carboxylic acids. Activated acids (e.g., esters with a good leaving group) can react with amines to form amides for example, whereas a normal carboxylic acid would just form a salt with an amine. EDAC can be used in combination with N-hydroxysuccinimide (NHS) for the immobilization of large biomolecules. NHS can be added to enhance crosslinking efficiency, and also aid with stabilizing the intermediates in the crosslinking process.

Referring again to FIG. 8, in various embodiments, method 800 can further comprise crosslinking the collagen-containing composition and the polymer in the second composition with EDAC and NHS (at step 880) and ethanol, at step 890, to form the bioscaffold structure. Ethanol can act as a buffer to stabilize the composition at an optimal pH range.

In accordance with various embodiments, an optimal pH range for the crosslinking reaction is about 4.0 to about 6.0. In accordance with various embodiments, the optimal pH range for the crosslinking reaction is about 5.3 to about 5.5. Moreover, the amount of EDAC and NHS used can depend, for example, on the components making up the collagen-containing composition. For example, while a given amount of EDAC and NHS can be used for a given amount of collagen (based primarily on the collagen concentration, and associated carboxyl groups associated with the collagen), that amount of EDAC and NHS can be increased if, for example, ECMs (e.g., elastin) are also included in the collagen-containing composition. A sufficient molar ratio for EDAC:NHS:carboxyl groups of collagen can be 5:5:1 for collagen-containing composition free of ECMs. Thus, EDAC and NHS can be added in excess to account for the addition of ECMs into the collagen-containing composition.

In accordance with various embodiments, the various methods can further comprise lyophilizing the formed bioscaffold structure. Such post-processing can serve, for example, to promote ease of storage and/or shipping.

As used in this specification and the appended claims, the singular forms “a,” “an,” and “the” include plural referents unless the content clearly dictates otherwise. As used in this specification and the appended claims, the term “or” is generally employed in its sense including “and/or” unless the context clearly dictates otherwise.

In light of the disclosure provided above, it will be apparent to those skilled in the art that various modifications and variations can be made in the practice of the various embodiments of the present disclosure without departing from the scope or spirit of the disclosure. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the disclosure will be apparent to those skilled in the art from consideration of the specification and practice of the various embodiments of the present disclosure.

It is noted in particular that where a range of values is provided in this specification, each value between the upper and lower limits of that range is also specifically disclosed. The upper and lower limits of these smaller ranges may independently be included or excluded in the range as well.

### EXEMPLIFICATION

### Example 1. Creation of the Bioscaffold

Natural or synthetic polymer constructs are manufactured using a bioprinter, such as a Celllnk (Palo Alto, Calif.) or RegenHU Bioprinter (RegenHU, Switzerland). The bioprinter is setup to allow for the printing of polymer via pneumatic driven syringes via a fused deposition modeler, which will allow for the deposition of molten polymer (FIG. 2). Polymer strands are deposited using a needle with a pressure of print pattern to create, for example, 50-1000 micron pores, with an angular range of 10-90 degrees to create a variety of shapes or configurations (FIG. 1).

Col-1 and Col-3 (Sigma Aldrich, MO or R&D Systems, MN) based bioinks, are deposited within the construct either in tandem (co-printed) in a similar fashion, or as a sequential layer (as a coating or surface modification following polymer deposition) (FIG. 3). Optionally, other bioactive molecules including, but not limited to, other types of collagen, extracellular matrix proteins, growth factors, cytokines, selected cell populations, small molecules and small molecule inhibitors, antibiotics, antimicrobials, nanoparticles, mesoporous silica, silk fibroin, and enzymatic degradation sites) are added in the same fashion.

Following printing, the construct and bioinks are cross-linked via CaCl2, ultraviolet (UV), or other methods. The tissue constructs have a maximal filament size of 100 μm, a final thickness of 0.5 mm to 20 cm, and may be printed to contain fenestrations and/or pores (FIG. 1). The solid, implant-style construct may be printed from a range of 20 g to 4500 g sizes for breast application or custom sized for other anatomic applications, and may be prolate spheroid shaped, custom shaped, or shaped in any other configuration (FIG. 1).

The construct contains a ratio of Col-1 to Col-3 of between 0.95:1±0.03 and 2.27±0.13, mimicking the Col-1 to Col-3 ratios contained within fetal and adolescent dermis. Mechanical properties for the final construct are within the following ranges: (a) tensile strength: 30-100 N/cm; (b) stiffness: <18 N/mm; (c) max load: >150N; (d) tensile stress: 10-30 MPa; (e) tensile strain: >35%; and (f) modulus of elasticity: <150 MPa.

1. An acellular matrix bioscaffold composition, comprising: 1) a natural or synthetic polymer or blend of polymers; 2) collagen I (Col-1); and 3) collagen III (Col-3), wherein the ratio of Col-1 to Col-3 is between about 0.5 and about 3.5, wherein said bioscaffold is synthetic.

2. The composition of claim 1, wherein the ratio of Col-1 to Col-3 is between about 0.9 and about 2.3.

3. The composition of claim 2, wherein the ratio of Col-1 to Col-3 is about 1.

4. The composition of claim 1, wherein the polymer is selected from the group consisting of poly(ethylene glycol) (PEG), poly(lactide-co-clycolide) (PLGA), polycaprolactone (PCL), poly(l-lactic acid) (PLLA), alginate, hyaluronic acid, gelatin, soy protein, fibrinogen, chitosan, dextran, and starch.

5. The composition of claim 1, wherein the polymer is polycaprolactone (PCL).

6. The composition of claim 1, further comprising pro-angiogenic bioactive molecules.

7. The composition of claim 1, further comprising anti-fibrotic molecules.

8. The composition of claim 1, further comprising cytokines.

9. The composition of claim 1, wherein the polymer is a blend of polymers.

10. An acellular matrix bioscaffold composition, comprising: 1) a natural or synthetic polymer; 2) collagen I (Col-1); 3) collagen III (Col-3), and 4) polycaprolactone (PCL), wherein said bioscaffold is synthetic and the ratio of Col-1 to Col-3 is between about 0.5 and about 3.5.

11. The composition of claim 10, further comprising an antimicrobial agent.

12. The composition of claim 10, further comprising a growth factor, cytokine or other bioactive molecule.

13. The composition of claim 10, further comprising pro-angiogenic bioactive molecules.

14. The composition of claim 10, further comprising anti-fibrotic molecules.

15. An acellular bioscaffold composition, comprising: 1) a polymer, 2) collagen I (Col-1), and 3) collagen III (Col-3), wherein the ratio of Col-1 to Col-3 is between about 0.9 and about 2.3, with the proviso that the bioscaffold does not comprise the bio-active molecules present in cadaveric acellular dermal matrix (ADM).

16. The composition of claim 15, further comprising an antimicrobial agent.

17. The composition of claim 15, further comprising a growth factor, cytokine or other bioactive molecule.

18. The composition of any one of claim 15, further comprising extracellular matrix proteins selected from the group consisting of laminin, fibronectin, elastin, glycosaminoglycans, and combinations thereof.

19. The composition of claim 15, wherein the bioscaffold comprises pores of about 100 to about 500 microns.

20. The composition of claim 15, wherein the bioscaffold comprises pores of about 50 to about 1000 microns.

21. The composition of claim 15, further comprising pro-angiogenic bioactive molecules.

22. The composition of claim 15, further comprising anti-fibrotic molecules.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)
